(19) United States
(12) Patent Application Publication
(10) Pub. No.: US 2022/0409379 A1
(43) Pub. Date: Dec. 29, 2022

(54) MEDICAL IMPLANT AND ANCHORING SYSTEM FOR A MEDICAL IMPLANT

(71) Applicant: FORMAE, INC., Paoli, PA (US)

(72) Inventor: Kevin A. Mansmann, Paoli, PA (US)

(73) Assignee: FORMAE, INC., Paoli, PA (US)

(21) Appl. No.: 17/781,558

(22) PCT Filed: Dec. 7, 2020
(86) PCT No.: PCT/US2020/063539

§ 371 Date: Jun. 1, 2022

Related U.S. Application Data
(60) Provisional application No. 62/944,467, filed on Dec. 6, 2019.

(51) Int. Cl. A61F 2/30; A61F 2/46

(52) U.S. Cl. CPC A61F 2/30749; A61F 2/4618; A61F 2002/30011; A61F 2002/30751; A61F 2002/30761; A61F 2002/30769; A61F 2002/3092; A61F 2002/3093; A61F 2002/30957; A61F 2002/30971; A61F 2002/4627; A61F 2002/4681

(57) ABSTRACT

A device configured for use as a medical implant is disclosed herein. The device includes an anchor body having a perimeter wall defining a rim, and a cavity dimensioned to receive an elastic articulating component. At least one lattice region is arranged at least along an inner surface of the perimeter wall adjacent to the rim. An elastic articulating component is configured to fill the cavity and attach to the at least one lattice region.

### INCORPORATION BY REFERENCE

This application claims the benefit of U.S. Provisional Patent Application 62/944,467, filed on Dec. 6, 2019, which is incorporated herein by reference as if fully set forth herein.

### FIELD OF INVENTION

The present invention relates to medical implants, and is more particularly related to medical implants and anchoring systems for medical implants configured to secure cartilage-like material to a bone.

### BACKGROUND

As explained in U.S. Patent Publication No. 2018/0289493, which is commonly owned by the present Applicant (Formae, Inc.), the entire contents of which is incorporated by reference as if fully set forth herein, medical implants including a hollow anchor having a cavity-like recess or receiving area with a raised or surrounding sidewall, rim, ridge or lip, for attaching a cartilage-like flexible material to a bone are known.

Cartilage is a flexible and relatively soft biological material that generally protects relatively hard bone, especially in the area of joints where bone is likely to be contacted by other hard surfaces. Natural cartilage forms a thin layer, usually in the range of 1-4 mm thick, which covers certain bone surfaces. Over time, cartilage deteriorates and becomes damaged due to use or other conditions. This is especially a problem of hyaline cartilage, a material that is found in articulating joints, including knees, hips, and shoulders in humans. For a variety of reasons, such cartilage is not as self-sustaining as other tissues, which leads to a need for repair and/or prosthetic replacement procedures, especially in the elderly.

As explained in U.S. Pat. No. 8,858,632, the entire contents of which is incorporated by reference as if fully set forth herein, hyaline cartilage is the main type of cartilage that provides smooth, slippery, lubricated surfaces that slide over and rub against other cartilage surfaces in “articulating” joints, such as knees, hips, shoulders, etc. Natural hyaline cartilage forms as a relatively thin layer (usually no more than about 3 or 4 millimeters thick) that covers certain surfaces of hard bones. While the hyaline cartilage in some joints (such as fingers) is not heavily stressed, the hyaline cartilage in other joints (notably including knees and hips) is frequently and repeatedly subjected to relatively heavy compressive loads, shear forces, and other stresses. Such cartilage does not have a blood supply or cellular structure that enables the type of cell turnover and replacement that occurs in many other tissues. As a result of those and other factors, hyaline cartilage in knees and hips may need repair or prosthetic replacement at fairly high rates among the elderly (due to gradual wear, injury, disorders such as osteoarthritis or rheumatoid arthritis, etc.), and at lower but considerable rates among younger patients (due to injury, congenital joint displacements that lead to unusual wear patterns, etc.).

It is important to recognize that hyaline cartilage is present only in relatively thin layers that coat the surfaces of bones. Since it is a soft tissue that cannot repair itself, it is vulnerable to damage when subjected to repeated loadings and stresses.

A bone surface that is covered by a layer of hyaline cartilage may be referred to herein as a “condyle.” However, it should be noted that this term is not always used consistently, by physicians and researchers. Some users limit “condyles” to the rounded ends of elongated bones. This usage includes the long bones in the arms and legs; it usually but not always includes smaller elongated bones in the hands, fingers, feet, and toes; and it normally excludes the cartilage-covered “sockets” in the ball-and-socket joints of the hips and shoulders (while encompassing the complementary ball ends of the other bone that fits such a socket). By contrast, other authors use “condyle” to refer to any bone surface covered by hyaline cartilage, including the socket surfaces in hip and shoulder joints. Since reinforced hydrogels as disclosed herein can be used to replace hyaline cartilage segments on any bone surface, the broader definition (which covers any bone surface covered by hyaline cartilage, including long bones, finger joints, socket surfaces in hips and shoulders, etc.) is used herein.

A condylar surface (i.e., a hyaline cartilage-carrying bone surface) contains a transition zone, called the subchondral layer or zone, at the interface between the hard bone and the cartilage. This transition zone strengthens and reinforces the cartilage, ensuring that the cartilage (which is relatively soft) is not readily pushed or scraped off the supporting bone when a joint is subjected to loading and shearing stresses. In the transition zone, large numbers of microscopic collagen fibers, firmly anchored in the hard bone, emerge from the bone in an orientation that is generally perpendicular to the bone surface at that location.

Bone is a relatively rigid biological material compared to cartilage. There are different typical rigidities of bones in the functional skeleton, corresponding to a large extent to the mechanical demands of the segment of bone, as outlined by Wolff s Law. Subchondral bone, the bone directly adherent to a cartilage layer at the joint surface, is comprised of a thin dense layer of bone. Less dense woven bone supports the subchondral joint articular surface. Dense cortical bone is found in the long bones for structural support.

Meniscal cartilage refers to specialized arc-shaped segments that help stabilize the knee and shoulder joints. Like hyaline cartilage (and unlike elastic cartilage or spinal cartilage), menisci have smooth lubricated surfaces that slide and rub against other cartilage surfaces, when a joint is articulating. They are made of a highly fibrous form of cartilage. Implants to replace meniscal cartilage are affixed to hard bone mainly via long fibers that extend out of the tips of the arcs, while the peripheral surfaces of the arcs are affixed to soft tissues instead of bone. In the shoulder joints, these arc segments are called labrum (or labral) segments; however, since their shapes and structures are nearly identical to meniscal segments in knees, and since labral cartilage in shoulders need to be repaired only rarely compared to meniscal cartilage in knees, labral cartilage is here included in the definition of the term “meniscal cartilage.”

Because of their function to bear lateral loading around arc shapes, meniscal cartilage segments have roughly triangular cross-sections, and their center regions have greater thickness than the hyaline cartilage layers that cover the surfaces of bones in joint regions.

The present disclosure relates to certain specific techniques and structural designs for anchoring devices and systems to fix hydrogel components of implants. Natural hyaline cartilage is present only in relatively thin layers that coat the surfaces of bones and diffuse into the bone tissue for affixation to the bone.

Most hydrogels that have substantial tensile strength, which are the hydrogels of most interest herein, hold water molecules within a cohesive polymeric molecular matrix, in a way that enables migration and diffusion of the water molecules through the molecular matrix. Although such hydrogel materials have at least some degree of deformability for purposes of elasticity, they cannot be in liquid form, i.e., they advantageously return to a specific non-deformed shape after loads or stresses have been removed.

For the purposes of this disclosure, synthetic hydrogel polymers are advantageously flexible, and can be rolled into cylindrical forms that can be inserted into a joint that is being surgically repaired, via a minimally invasive incision, using an arthroscopic insertion tube. By avoiding and eliminating the need for “open joint” surgery, arthroscopic insertion of a flexible implant in a rolled-up cylindrical form can spare surrounding tissues and blood vessels from more severe damage during an open joint surgical operation.

Due to these and other factors, hydrogel materials are able to provide better performance than the solid plastics, such as ultra-high molecular weight polyethylene (“UHMWPE”) that are used today in many hip and knee replacements.

In natural cartilage, the hydrogel structure is created by a three-dimensional matrix that is given shape and strength mainly by collagen. Collagen is a fibrous protein that holds together nearly all soft tissues in animals. In synthetic hydrogels, the three-dimensional matrix usually has a molecular structure made of complex polymers that have a combination of: (i) long continuous chains (i.e. “backbone” chains), containing mainly carbon atoms and sometimes containing oxygen, nitrogen, sulfur, or other atoms as well; (ii) side chains, which branch off the backbone chains in ways that can have either controlled or semi-random spacing, length, content, etc.; and (iii) crosslinking bonds, which connect the backbone and side chains to each other in ways that create complex three dimensional molecules that have sufficient spacing between them to allow water molecules to travel within the molecular matrix.

Synthetic hydrogel polymers advantageously are hydrophilic, to cause them to attract and hold water molecules. This can be accomplished by including large numbers of oxygen atoms (usually in hydroxy groups), nitrogen atoms, or other non-carbon atoms in the backbone and/or side chains, to provide “polar” groups that will attract water, which is a polar liquid.

Fluid permeability (which involves the ability of water to pass through the molecular matrix of cartilage) is important in the behavior and performance of natural cartilage. As an example, FIG. 6 in U.S. Pat. No. 6,530,956, which is incorporated herein by reference as if fully set forth herein, illustrates how fluid flow through cartilage can help distribute stresses and pressures that are imposed on cartilage in a load-bearing joint such as a knee, when a person is walking or running.

Due to these and other factors, hydrogel materials are of interest in joint repair implants, and may be able to provide better performance than the solid plastics (such as high molecular weight polyethylene, abbreviated as UHMWPE) that are used today in most hip and knee replacements.

There are ongoing efforts to provide improved hydrogel implants for replacing cartilage in joints are described in the present inventor's patents and patent applications such as U.S. Pat. No. 6,629,997 (“Meniscus-Type Implant With Hydrogel Surface Reinforced By Three-Dimensional Mesh”), U.S. Pat. No. 9,050,192 (“Cartilage Repair Implant With Soft Bearing Surface And Flexible Anchoring Device”), U.S. Patent Pub. No. 2002/0183845 (“Multi-Perforated Non-Planar Device For Anchoring Cartilage Implants And High-Gradient Interfaces”), U.S. Pat. No. 9,314,339 (“Implants For Replacing Cartilage, With Negatively-Charged Hydrogel Surfaces And Flexible Matrix Reinforcement”), all of which are hereby incorporated by reference in their entireties as though fully set forth herein.

To employ soft hydrogel in an implant to replace damaged cartilage, it is advantageous to anchor the hydrogel to the associated bone articulating surface in such a way as to promote healing of the hydrogel implant to the bone recipient site, i.e., to secure the implant that carries the hydrogel surface exposed for sliding articulation. There is a significant modulus of elasticity mismatch in structural characteristics between the cartilage, with relatively soft fragile material properties, and the subchondral bone, with relatively tough rigid material properties.

Because hydrogel polymers (which contain substantial quantities of water molecules) will inevitably be weaker than various known types of hard plastics that do not contain any free water, the work by the present inventor, has focused on hydrogels that are reinforced by three-dimensional fiber arrays, made of synthetic fibers having high tensile strength.

Difficulty in anchoring these composite fiber reinforced hydrogels to bone has been encountered, with the difficulty centered at the hydrogel anchor interface and the bone anchor interface.

### SUMMARY

A device configured for use as a medical implant is disclosed herein. The device includes an anchor body having a perimeter wall defining a rim, and a cavity dimensioned to receive an elastic articulating component. At least one lattice region is arranged at least along an inner surface of the perimeter wall adjacent to the rim. An elastic articulating component is configured to fill the cavity and attach to the at least one lattice region.

In one aspect, the at least one lattice region includes a first lattice region and a second lattice region that are distinct from each other. The first lattice region is arranged along a bottom region of the perimeter wall and the second lattice region is arranged along the inner surface of the perimeter wall adjacent to the rim.

In one aspect, the perimeter wall defines an inner surface between the first lattice region and the second lattice region, and the inner surface also includes at least one non-lattice region within the cavity.

The first lattice region may include a plurality of struts extending upward from a bottom surface of the perimeter wall. In one aspect, a height of the plurality of struts increases in a direction starting from the perimeter wall and radially towards a center of the anchor body. A top surface of the first lattice region may define a radius of curvature between diametrically opposed surfaces of the perimeter wall.

In one aspect, the first lattice region has a radially innermost edge that is arranged radially outward from an aperture defined by a partially closed bottom surface of the perimeter wall.

In one aspect, the first lattice region and the second lattice region are radially offset from each other relative to a central axis of the anchor body.

The elastic articulating component is preferably molded with the anchor body, and the elastic articulating component completely surrounds both the first lattice region and the second lattice region. The elastic articulating component is preferably formed from hydrogel.

An outer porous layer is arranged on an outer surface of the perimeter wall in one aspect.

In one aspect, the at least one lattice region is defined along an entirety of the inner surface of the perimeter wall. In another aspect, the at least one lattice region is only defined in a region directly adjacent to the rim. In one aspect, the at least one lattice region is defined as an annular region arranged in a medial area along the interior surface of the perimeter wall, and spaced away from the rim and a bottom surface of the anchor body.

Additional aspects and embodiments are described herein.

### BRIEF DESCRIPTION OF THE DRAWINGS

The foregoing Summary and the following detailed description will be better understood when read in conjunction with the appended drawings, which illustrate a preferred embodiment of the invention. In the drawings:

FIG. 1A is a front view showing the interface between a femur and tibia and the location of the implant

FIG. 1B is a perspective view showing the interface between a femur and tibia and the location of the implant.

FIG. 2A is a top view of an anchor body according to one aspect.

FIG. 2B is a front perspective view of the anchor body of FIG. 2A.

FIG. 2C is a front elevated perspective view of the anchor body of FIGS. 2A and 2B.

FIG. 3A is a side view of the implant including an articulating component attached to the anchor body according to an aspect.

FIG. 3B is a top perspective view of the implant of FIG. 3A.

FIG. 3C is an elevated perspective view of the implant of FIGS. 3A and 3B.

FIG. 4A is an elevated perspective view of an anchor body according to an aspect.

FIG. 4B is a partial cross-section side view of the anchor body of FIG. 4A.

FIG. 4C is an elevated partial cross-section view of the anchor body of FIGS. 4A and 4B.

FIG. 5A is a side cross-section view of an implant having a first size.

FIG. 5B is a side cross-section view of an implant having a second size.

FIG. 5C is a side cross-section view of an implant having a third size.

FIG. 6 is a side view of the implant in situ.

FIG. 7A is a side cross-sectional view of an implantation tool engaging an implant according to one aspect.

FIG. 7B is an alternative side cross-sectional view of the implantation tool of FIG. 7A.

FIGS. 7C and 7D are perspective views of the implantation tool of FIGS. 7A and 7B in varying states.

FIG. 7E is a cross-sectional of the implantation tool of FIGS. 7A-7D.

FIG. 8 is a cross-sectional view of an assembly for forming an implant according to one aspect.

FIG. 9A is an elevated partial cross-section view of another aspect of an anchor body.

FIG. 9B is an elevated partial cross-section view of another aspect of an anchor body.

FIG. 9C is an elevated partial cross-section view of another aspect of an anchor body.

FIG. 9D is an elevated partial cross-section view of another aspect of an anchor body.

FIG. 9E is an elevated partial cross-section view of another aspect of an anchor body.

### DETAILED DESCRIPTION OF THE PREFERRED EMBODIMENTS

The description provided herein is to enable those skilled in the art to make and use the described embodiments set forth. Various modifications, equivalents, variations, combinations, and alternatives, however, will remain readily apparent to those skilled in the art. Any and all such modifications, variations, equivalents, combinations, and alternatives are intended to fall within the spirit and scope of the present invention defined by claims.

Certain terminology is used in the following description for convenience only and is not limiting. The words “right,” “left,” “top,” and “bottom” designate directions in the drawings to which reference is made. The words “a” and “one,” as used in the claims and in the corresponding portions of the specification, are defined as including one or more of the referenced item unless specifically stated otherwise. This terminology includes the words above specifically mentioned, derivatives thereof, and words of similar import. The phrase “at least one” followed by a list of two or more items, such as “A, B, or C,” means any individual one of A, B or C as well as any combination thereof.

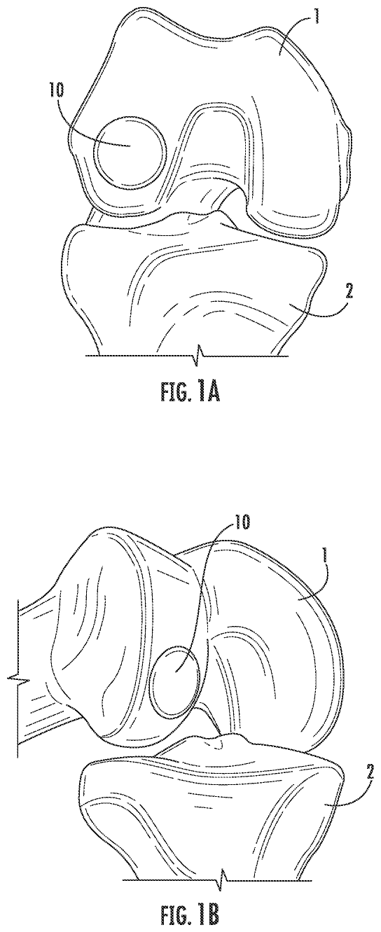

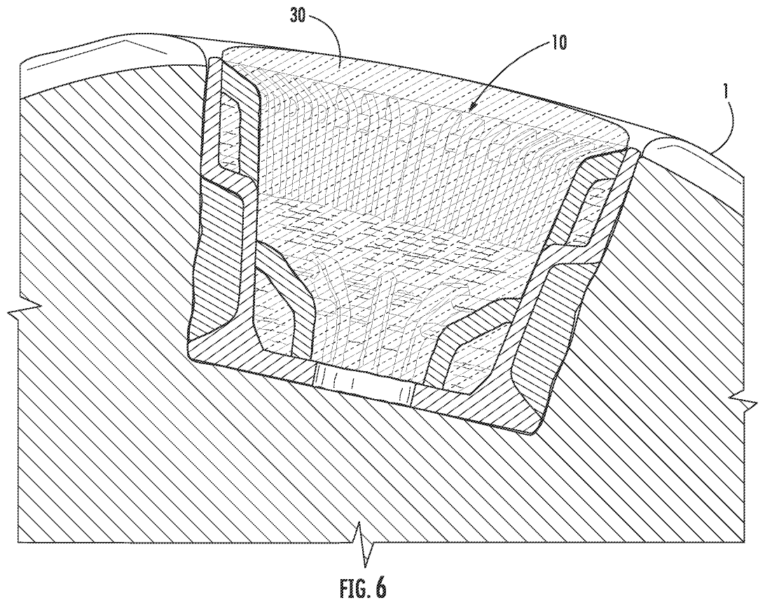

As shown in FIGS. 1A and 1B, the implant 10 disclosed herein is configured to be installed within a patient's bone, and particularly a patient's femur 1, and more specifically on an end of the femur 1 that engages the tibia 2. FIG. 6 illustrates the implant 10 in situ or fully installed in the femur 1. Further details regarding the placement of the implant 10 are disclosed in U.S. Patent Publication No. 2018/0289493, the entire contents of which are incorporated by reference as if fully set forth herein.

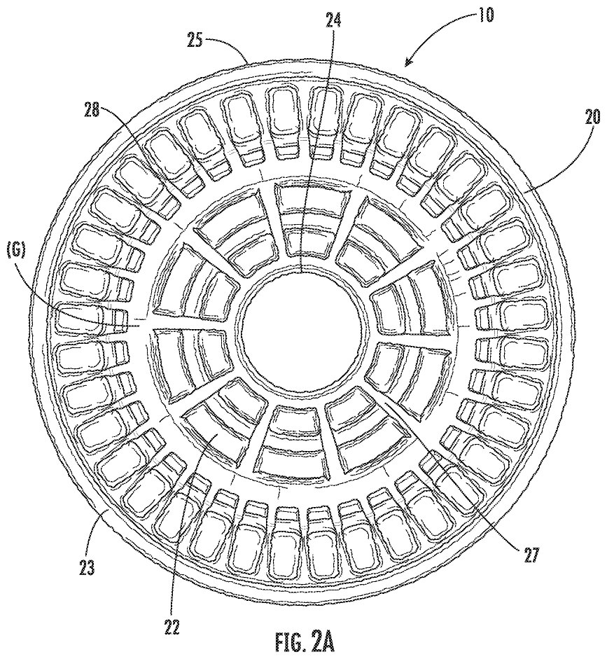

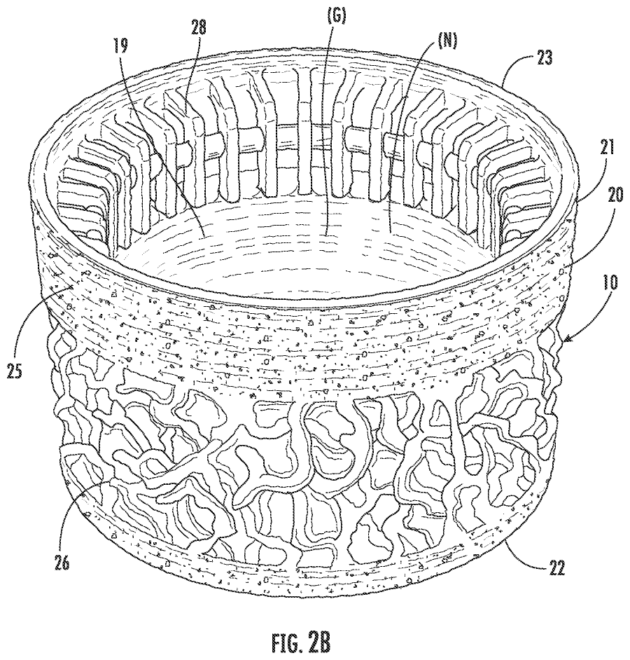

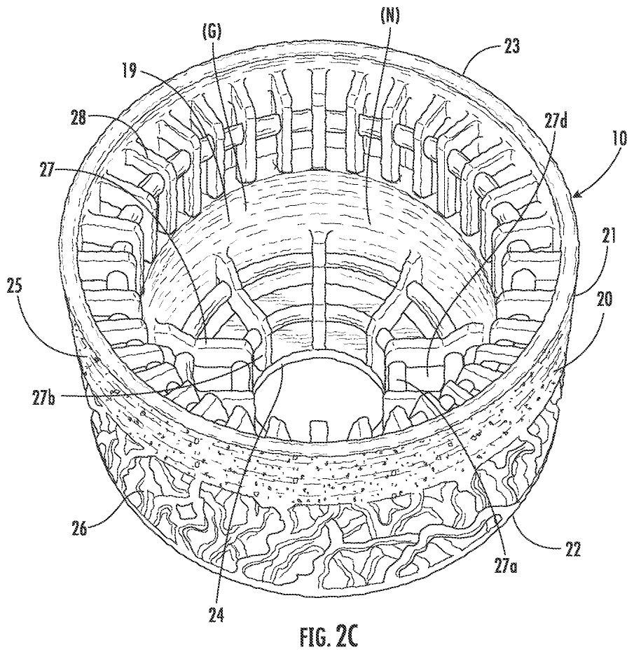

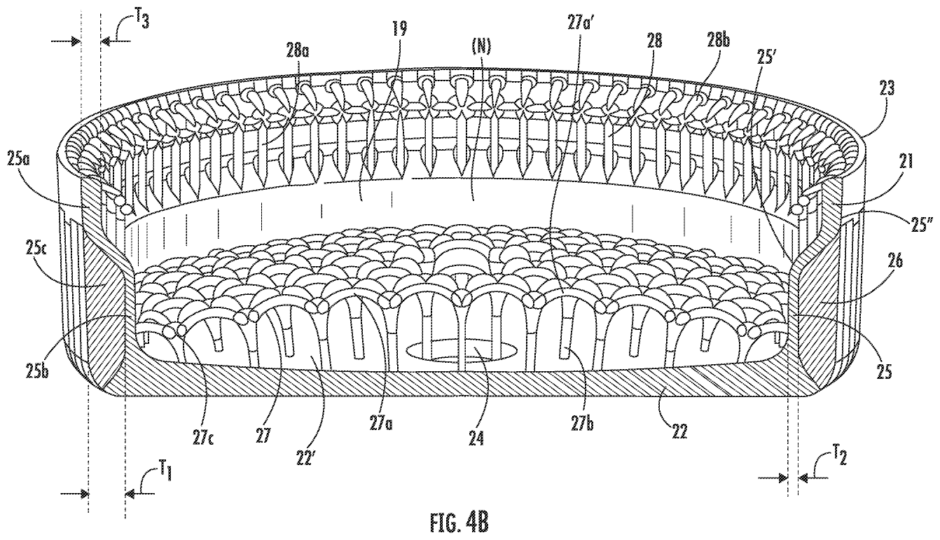

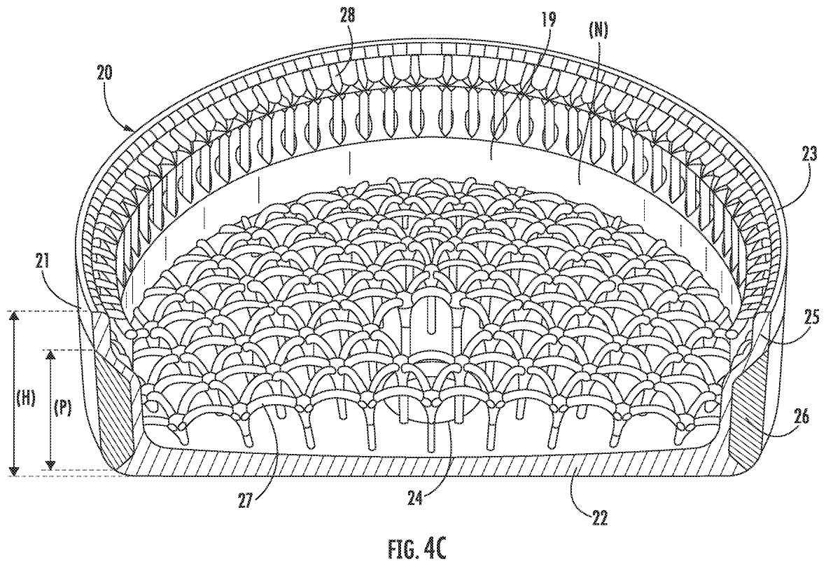

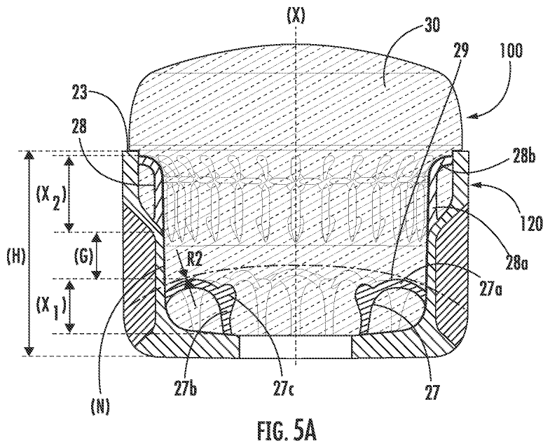

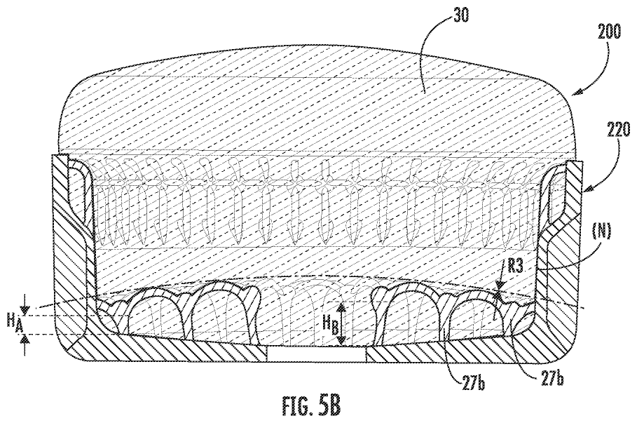

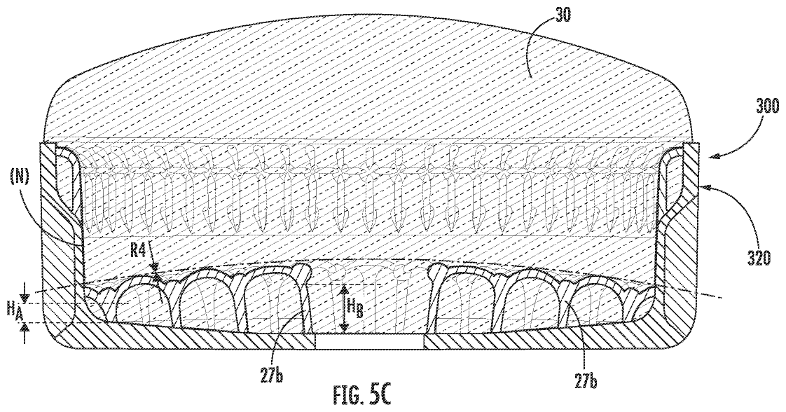

In general, the implant 10 and its components are shown in a photorealistic style in FIGS. 2A, 2B, 2C, 3A, 3B, 3C, and 6, and the implant 10 and its components are shown in an engineering schematic representation in FIGS. 4A, 4B, 4C, 5A, 5B, and 5C. All of the Figures generally illustrate the same component unless specific variations are identified herein.

As shown in FIGS. 2A-2C, the implant 10 includes an anchor body 20. The anchor body 20 includes at least two lattice regions. In one aspect, a first lattice region 27 is disposed or positioned on or at a base or bottom wall 22 of the anchor body 20, and a second lattice region 28 is disposed or positioned along an inner surface of a sidewall or perimeter wall 25. A gap (G) preferably separates the first lattice region 27 and the second lattice region 28 from each other. In one aspect, the second lattice region 28 is arranged adjacent to a rim 23 of the perimeter wall 25.

Therefore, it is contemplated that there are regions of the anchor body that include one or more lattice regions, such as regions 27 and 28, and regions that are void of any lattice, i.e. non-lattice regions (indicated with reference annotations (N) and (N′) in the FIGS. 2B, 2C, 3B, 3C, 4A-4C, 5A-5C, and 9A-9E) or barrier layer only regions. Regions or areas of the anchor body within the cavity that do not include the lattice can be considered to be non-lattice regions such that the barrier layer is directly exposed and considered the surface layer.

One of ordinary skill in the art would understand that the anchor body 20 can be formed according to a plurality of various profiles. For example, the anchor body 20 can include a curved sidewall or perimeter wall and a flat base or bottom wall. In another aspect, the anchor body 20 can include a generally curved profile, such as a half-spherical profile. Regardless of the shape, the anchor body 20 includes distinct lattice regions that are spaced apart from each other according to one aspect of the invention.

It is appreciated that the lattice regions may comprise a frame, framework, matrix, web, mesh, or other arrangements whereby a structure having openings is provided with at least some attachment or anchoring areas around the openings. In one aspect, the lattice regions, as well as a remainder of the anchor body, are formed by 3D printing techniques.

In one aspect, the perimeter wall 25 generally includes an inner barrier layer 25′ (shown in FIG. 4B) that may have a lower or lesser porosity than the porosity of an outer layer 26, no porosity, or essentially no porosity, and an outer porous layer 26 which is configured to optimize bone ingrowth, as described in U.S. Patent Publication No. 2018/0289493. The inner barrier layer 25′ of the perimeter wall 25 may be solid or generally impermeable, such as impermeable to the elastic articulating component 30 (i.e. the flexible material or hydrogel), while the outer layer 25″ of the perimeter wall 25 (including outer layer 26 in one aspect) is substantially or generally porous to promote bone ingrowth. This design ensures that an elastic articulating component 30, e.g. a flexible material such as hydrogel, is contained and/or maintained within a cavity 19 defined by the perimeter wall 25 and the base or bottom wall 22 during an assembly step in which the articulating component 30 is injected into the anchor body 20, as well as during placement or use in some instances. The barrier layer 25′ acts as a barrier that the elastic articulating component 30 cannot pass through. In one aspect, the entire perimeter wall 25 can be formed as a barrier layer or barrier wall. The elastic articulating component 30 completely fills the cavity 19 in one aspect.

As shown in FIG. 4A, a central area 22′ of the bottom wall 22, comprises an aperture 24 or opening in which the first lattice region 27 is absent. A size of this aperture 24 can vary. Except for this aperture 24, the first lattice region 27 preferably covers an entirety of the base of the anchor body 20. In different aspects, the bottom wall 22 does not include the aperture 24. In other aspects, the first lattice region 27 extends over the aperture 24.

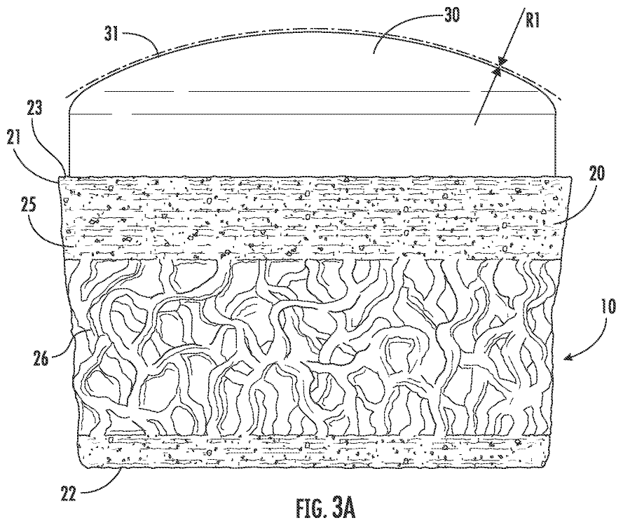

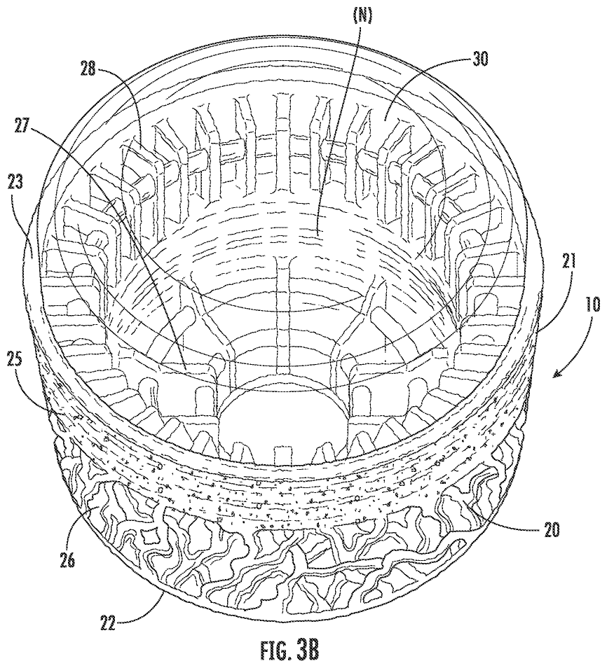

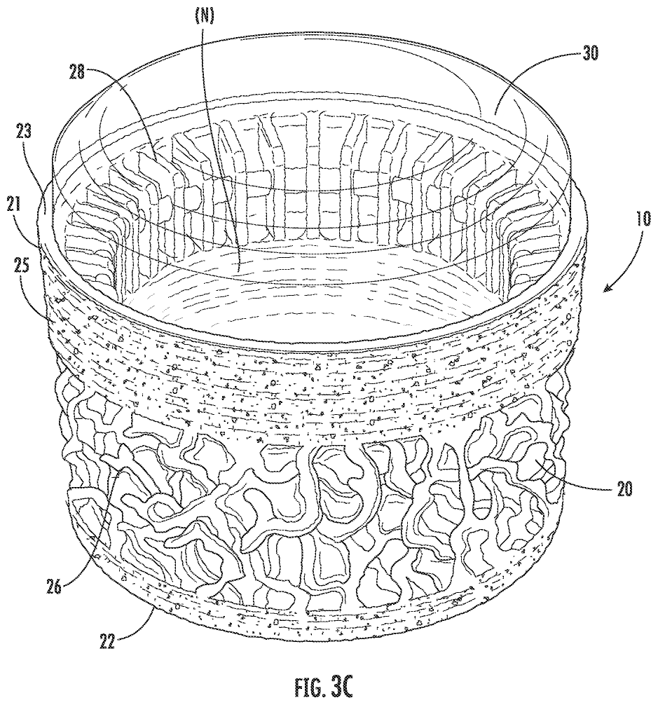

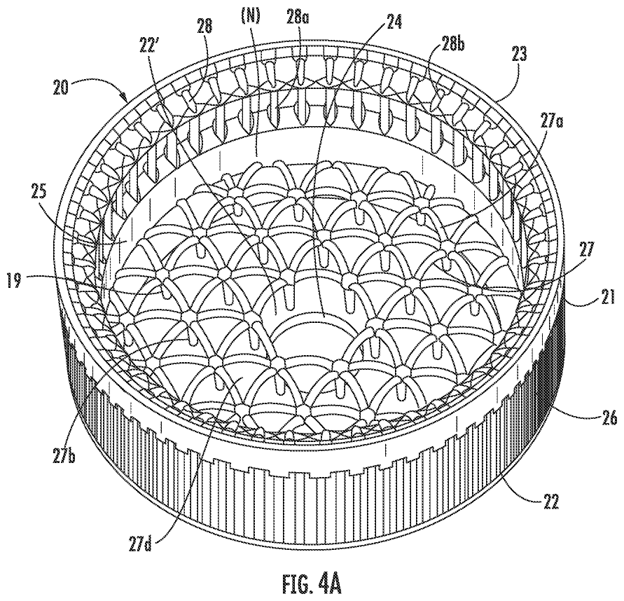

As shown in FIGS. 3A-3C, the implant 10 includes an elastic articulating component 30, formed from a flexible material such as a hydrogel. Further details of materials that may form the articulating component 30 are provided in U.S. Patent Publication No. 2018/0289493, in which the articulating component is generally disclosed as a “flexible material 14.”

In one aspect, the articulating component 30 is preferably formed from a hydrogel, and comprises a polymeric molecular matrix that cohesively holds water molecules. The articulating component 30 has some degree of deformability and is elastic such that the articulating component 30 returns to a specific non-deformed shape after loads or stresses are removed. More specifically, the articulating component 30 is formed of hydrogel that consists of a three-dimensional matrix having a molecular structure made of complex polymers. The articulating component 30 is hydrophilic in one aspect. Specifically, the articulating component 30 includes a relatively high proportion of oxygen atoms (i.e. in hydroxy groups), nitrogen atoms, or other non-carbon atoms to provide “polar” groups that attract water.

FIG. 3A illustrates the articulating component 30 has a top surface 31 defining a radius of curvature (R1). This radius of curvature (R1) is configured to match, conform to, correspond to, or mimic a patient's anatomy, i.e. a curvature of a patient's femur 1 in a region of the implantation site, as shown in FIGS. 1A, 1B, and 6. In one aspect, the radius of curvature (R1) is 15 mm-40 mm.

Further details of the anchor body 20 are provided herein and are illustrated in FIGS. 4A-4C. The anchor body 20 has an upper end 21 including a rim 23 defining an opening dimensioned to receive the articulating component 30. In one aspect, the anchor body 20 includes a bottom wall or closure end 22 including a bottom surface 22′, which can define the aperture 24. One of ordinary skill in the art would understand that the shape of the anchor body 20 can vary greatly, and is dependent upon a specific implantations site and/or patient anatomy.

The perimeter wall 25 may be generally annular, cylindrical, spherical, curved, frusto-conical, or any geometric shape. The perimeter wall 25 preferably extends between the end 21 and the bottom wall 22, and the perimeter wall 25 includes an inner barrier layer or surface 25′, an outer surface 25″, and a medial region that tapers radially inward in a direction from the end 21 to the bottom wall 22, such that the outer surface 25″ of the perimeter wall 25 defines a pocket 25c. In one aspect, the inner surface 25′ of the perimeter wall 25 defines an inner barrier layer configured to restrict flow or seepage of the articulating component 30 during assembly, i.e. molding of the articulating component 30 into the already formed anchor body 20. In one aspect, the outer surface 25″ does not include a barrier layer and instead is configured to promote bony growth and includes pores.

The outer porous layer 26 is a bony growth region and is preferably arranged within the pocket 25c of the perimeter wall 25. The outer layer 26 is preferably a trabecular porous metal, or any material configured to optimize bone ingrowth, as understood in the field of orthopedic surgery.

A first thickness (t1) of the outer layer 26 is larger than a second thickness (t2) of the perimeter wall 25 in a region of the outer layer 26. In one embodiment, the first thickness (t1) of the outer layer 26 is at least three times larger than a second thickness (t2) of the perimeter wall 25 in a region of the outer layer 26. The rim 23 of the anchor body 20 has a third thickness (t3) which is greater than the second thickness (t2) of the perimeter wall 25 in the region of the outer layer 26, and the third thickness (t3) is less than the first thickness (t1) of the outer layer 26. The pocket 25c and the outer layer 26 each generally have a generally trapezoidal profile.

The first lattice region 27 is arranged along the bottom surface 22′ adjacent to the inner surface 25′ of the perimeter wall 25. In one embodiment, the first lattice region 27 defines an upper surface 29 facing the end 21 of the anchor body 20, and the upper surface 29 is defined by a plurality of cross-struts 27a each defining curved bearing surfaces 27a′ configured to engage the articulating component 30. As shown in the drawings, the cross-struts 27a define convex bearing surfaces that curve upwards towards the rim 23. In one aspect, the cross-struts 27a have a straight or flat profile. As used herein, the term cross-strut refers to a post, beam, bar or other structure extending generally or partially perpendicular to a central axis (X) of the implant 10 (e.g., as illustrated shown in FIG. 5A). The first lattice region 27 includes a plurality of struts 27b positioned along the bottom surface 22′ that support the cross-struts 27a. As used herein, the term strut refers to a post or other structure extending generally parallel to the central axis (X) of the implant 10 (as illustrated shown in FIG. 5A). Holes 27d are defined by the spaces between the cross-struts 27a and the struts 27b. In one aspect, as shown in FIG. 4A, the holes 27d defined between the cross-struts 27a have a non-circular profile. In one aspect, the holes 27d have a generally triangular profile. One of ordinary skill in the art would recognize based on this disclosure that the shape and profile of the holes 27d can vary.

The struts 27b and the cross-struts 27a intersect with each other at a plurality of junction regions 27c. The junction regions 27c comprise the areas where ends of multiple cross-struts attach to the same strut. In one aspect, a majority of the junction regions 27c intersect with six cross-struts 27a and a single strut 27b, as shown in FIG. 4A. A thickness of the first lattice region 27 at the plurality of junction regions 27c is greater than a thickness of the first lattice region 27 at the struts 27b and the cross-struts 27a. In one embodiment, a height of the struts 27b increases in in a radially inward direction starting from the perimeter wall 25 and going inwards toward the aperture 24 defined in the bottom surface 22′.

A second lattice region 28 is arranged along the inner surface 25′ the perimeter wall 25 on the end 21. The second lattice region 28 is spaced apart from the first lattice region 27 by a gap (G). In other words, the perimeter wall 25 provides a smooth surface between the lattice regions 27, 28. The second lattice region 28 defines a flat vertical bearing surface via a plurality of struts 28a and a curved top surface defined by a plurality of cross-struts 28b extending from the struts 28a to the rim 23 of the anchor body 20.

The first lattice region 27 has a radially innermost edge that is arranged radially outward from the aperture 24 defined by the partially closed bottom wall 22 of the anchor body 20.

The gap (G) defined between the first lattice region 27 and the second lattice region 28 is preferably at least 10% of a total axial height (H) of the anchor body 20, and more preferably the gap (G) defined between the first lattice region 27 and the second lattice region 28 is at least 20% of a total axial height (H) of the anchor body 20. In an embodiment, the gap (G) between the first lattice region 27 and the second lattice region 28 is 10%-30% of the total axial height (H) of the anchor body 20.

The pocket 25c defined by the perimeter wall 25 has an axial extent (P) that is preferably at least 60% of a total axial height (H) of the anchor body 20, and, more preferably, the pocket 25c defined by the perimeter wall 25 has an axial extent (P) that is preferably at least 75% of a total axial height (H) of the anchor body 20. In an embodiment, the axial extent (P) of the pocket 25c is between 60%-90% of the total axial height (H) of the anchor body 20.

The first lattice region 27 has a first average axial height (X1) in a region of the perimeter wall (which gradually increases towards a radial center of the anchor body), and the second lattice region 28 has a second axial height (X2) that is greater than the first axial height (X1).

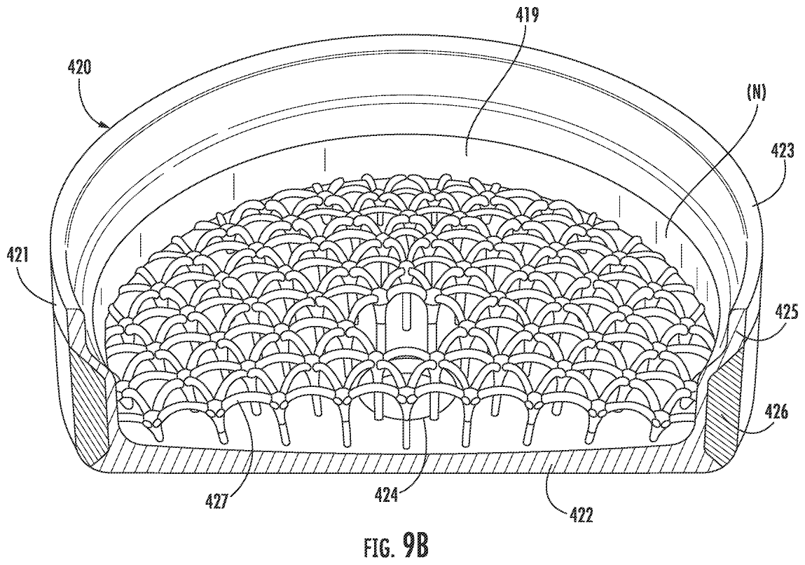

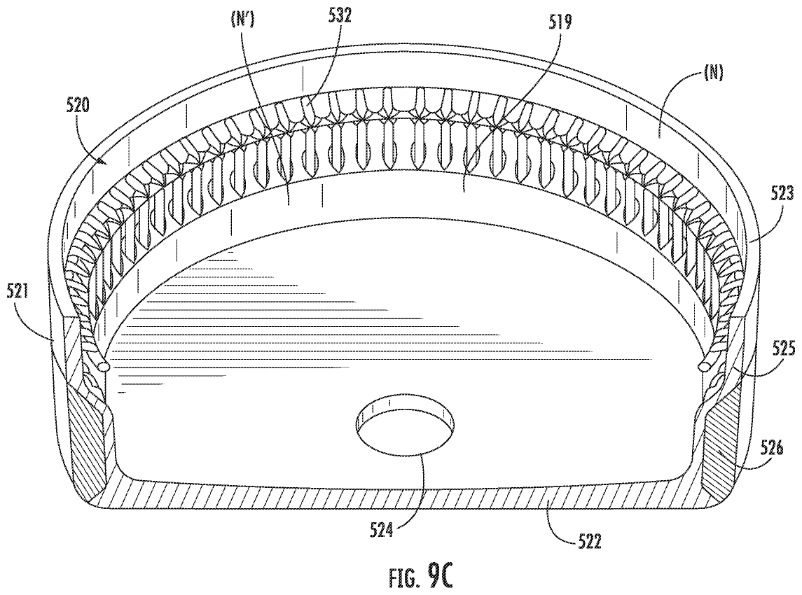

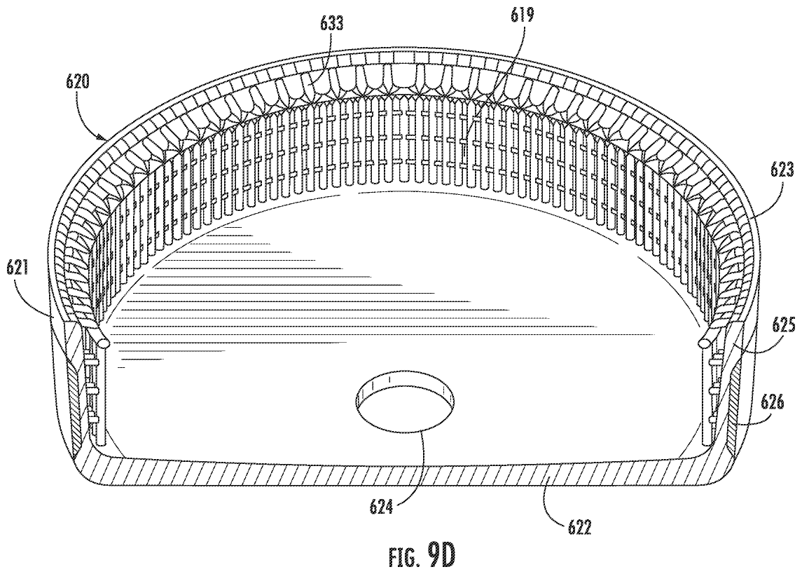

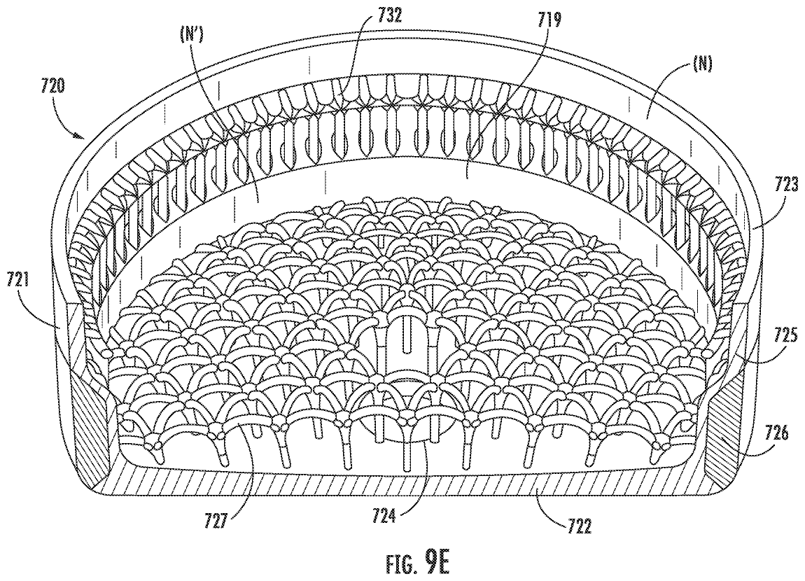

FIGS. 9A-9E illustrate various aspects of anchor bodies 320, 420, 520, 620, 720. Each of these anchor bodies 320, 420, 520, 620, 720 generally include the same structure or arrangement as the anchor body 20 unless specifically explained in more or different detail herein. In each of these aspects, one or more non-lattice regions (N, N′) are defined on an inner surface of the perimeter wall and defining the cavity.

As shown in FIG. 9A, in one aspect, the anchor body 320 only includes a lattice region 328 around a rim 323 adjacent an upper portion of the anchor body 320. In this aspect, the remainder of the cavity 319 does not include any lattice or attachment portions configured to engage the articulating component 30. Accordingly, the base or bottom wall 322 does not include any lattice regions and an interior surface of the perimeter wall 325 also does not include any lattice regions. The base or bottom wall 322 and an interior surface of the perimeter wall 325 may preferably include barrier layers.

FIG. 9B illustrates an aspect of the anchor body 420 in which a lattice region 427 is only defined in a base or bottom region 422 of the anchor body 420. In this aspect, the remainder of the cavity 419 does not include any lattice or attachment portions configured to engage the articulating component 30. Accordingly, the rim 423 does not include any lattice regions, and an interior surface of the perimeter wall 425 in regions spaced away from the base or bottom region 422 also does not include any lattice regions. As a result, the non-lattice region (N) in FIG. 9B is relatively larger than the non-lattice regions of other embodiments. In this embodiment, the top surface of the lattice region 427 includes a radius of curvature as described with respect to the other aspects of the lattice region 27. The base or bottom wall 422 and an interior surface of the perimeter wall 425 may preferably include barrier layers. It is noted that the lattice region 427 does not fully fill the cavity 419, but only fills a portion of the cavity 419.

FIG. 9C illustrates another aspect of the anchor body 520 in which a lattice region 532 is arranged in a medial area of the interior of the perimeter sidewall 525 such that the lattice region 532 is spaced apart from the rim 523 and also spaced apart from a bottom wall or bottom surface 522. In other words, the rim 523 and an area adjacent the rim 523, and the bottom surface 522 both do not include any lattice regions. Accordingly, two distinct and separate non-lattice regions (N, N′) are provided. The base or bottom wall 522 and an interior surface of the perimeter wall 525 may preferably include barrier layers.

FIG. 9D illustrates an aspect of an anchor body 620 in which an entirety of the sidewall of the anchor body 620 includes a lattice region 633. In this configuration, the lattice region 633 is provided in a region of the rim 623 and extends continuously axially downward to the bottom wall or bottom surface 622. The base or bottom wall 622 and an interior surface of the perimeter wall 625 may preferably include barrier layers.

FIG. 9E illustrates an aspect of an anchor body 720 in which a first lattice region 732 is defined in a medial area of the interior of the perimeter sidewall 725 such that the first lattice region 732 is spaced apart from the rim 723 and also spaced apart from a bottom wall or bottom surface 722. A second lattice region 727 is defined on the bottom wall or bottom surface 722, which is spaced apart from the first lattice region 732. Accordingly, in this aspect, two distinct and separate non-lattice regions (N, N′) are defined. The base or bottom wall 722 and an interior surface of the perimeter wall 725 may preferably include barrier layers.

As illustrated by FIGS. 9A-9E, lattice regions can be provided in a variety of configurations, shapes, sizes, locations, etc. It is appreciated that various configurations of the lattice regions can be provided, including combinations of all or portions of the rims, sidewalls, and bottom surfaces of the anchor bodies.

The gap (G), the first axial height (X1), the second axial height (X2), and the total axial height (H) are illustrated in FIG. 5A. FIGS. 5A-5C also illustrate variations of medical implants 100, 200, 300 including an anchor body 120, 220, 320 having varying dimensions, but the same essential features of the anchor body 20 of FIGS. 4A-4C.

As shown in FIG. 5A, the first lattice region 27 includes an upper surface 29 having a radius of curvature (R2) which is defined between diametrically opposed surfaces of the perimeter wall 25. This radius of curvature (R2) is generally defined by the collective curvature defined by uppermost point of the cross-struts 27a. In one aspect, heights of the struts 27b generally increases towards a center region of the anchor body. The radius of curvature (R₂) is 15 mm-30 mm in one aspect.

As shown in FIGS. 5B and 5C, the struts 27b have a smaller height (HA) in areas around an outer periphery of the cavity 19 and a larger height (HB) at areas towards a central region of the cavity 19. This feature is also generally shown in FIG. 4B. This variation in height causes the upper bearing surface of the cross-struts 27a to define the radius of curvature. In FIG. 5B, the radius of curvature (R3) is 20 mm-35 mm. In FIG. 5C, the radius of curvature (R4) is 25 mm-40 mm. One of ordinary skill in the art would understand that the radius of curvature of the lattice structures can vary depending on the specific application, size of the articulating component, and other factors.

In general, the radius of curvature defined by the top bearing surfaces of the first lattice regions 27 in each embodiment is selected to support the corresponding bearing surface defined by the top surface 31 of the articulating component or hydrogel 30, which is dictated based on patient anatomy. In one aspect, the radius of curvature defined by the top bearing surfaces of the first lattice regions 27 in each embodiment is selected to be identical to or within 1%-10% of a radius of curvature defined by the top surface 31 of the articulating component or hydrogel 30. In other words, the radius of curvature of the top bearing surfaces of the first lattice regions 27 in each embodiment is selected to essentially mimic a patient's anatomy.

Once the articulating component or hydrogel 30 is injected into the anchor body 20, the articulating component or hydrogel 30 fully molds around the first lattice region 27 and the second lattice region 28. In other words, the articulating component or hydrogel 30 flows or seeps such that the articulating component or hydrogel 30 is secured to the lattice regions 27, 28 via gripping.

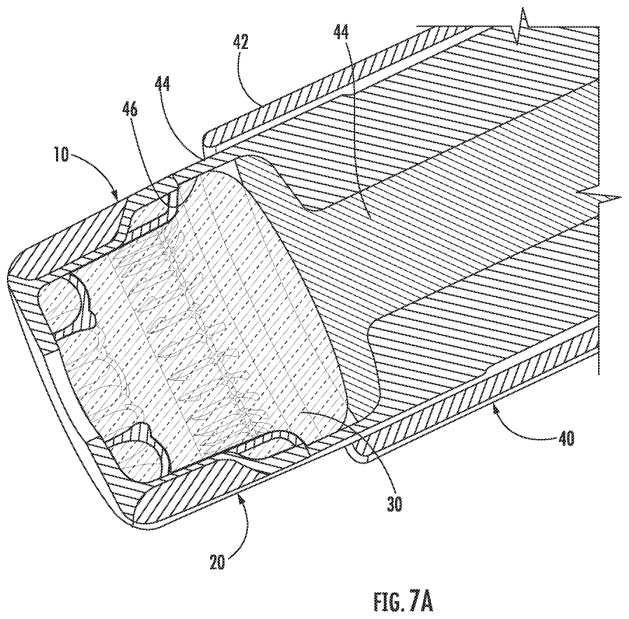

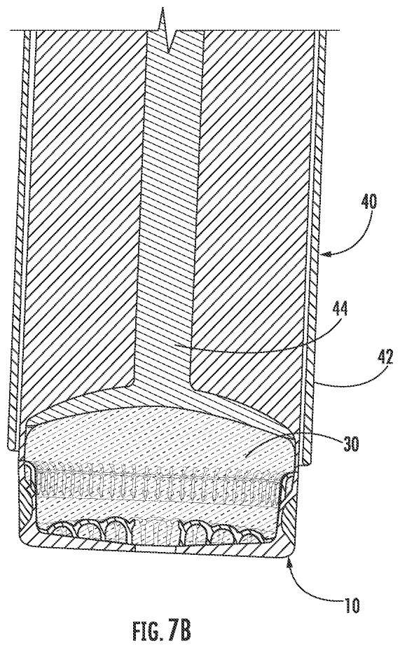

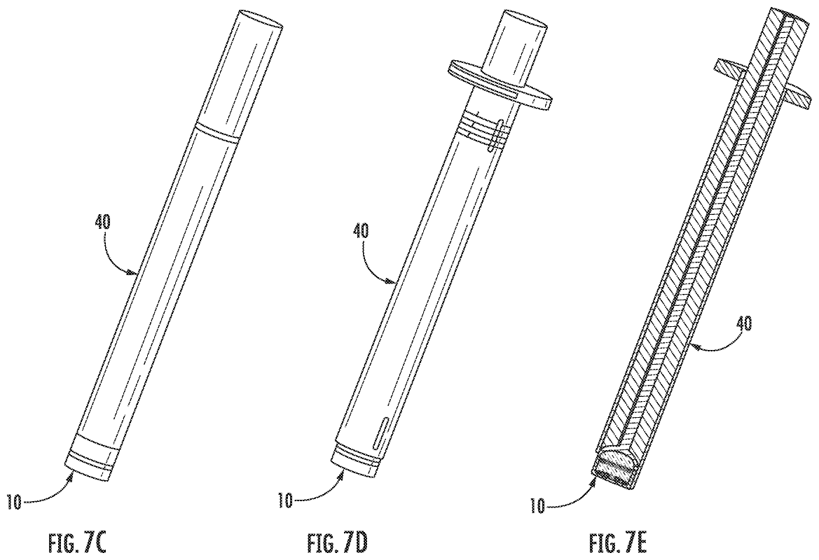

FIGS. 7A-7E illustrate a medical implant tool 40 for delivering the implant 10 into a bone, such as a femur 1. The implant tool 40 generally impacts the rim 23 of the anchor body 20 while protecting the articulating component 30 to secure the implant 10 in a bore of the femur 1 via a press fit. The medical implant tool 40 includes an outer sleeve 42, and a plunger 44 arranged inside of the outer sleeve 42 such that the plunger 44 is axially moveable relative to the outer sleeve 42. The plunger 44 defines a collar 46 dimensioned to receive a portion, i.e. the articulating component 30, of the medical implant 10.

A method of inserting the medical implant 10 using the medical implant tool 40 is also disclosed. The method includes providing the medical implant 10 and the medical implant tool 40, creating an opening in a patient's bone (i.e. the femur 1), inserting the medical implant 10 inside the opening in the patient's bone, and engaging the medical implant 10 with the medical implant tool 40, such that the collar 46 receives a portion of the articulating component 30, and the collar 46 engages the rim 23 defined at the end 21 of the anchor body 20. The medical implant tool 40 is used to forcefully impact the implant 10 into an undersized hole in the bone, without transmitting any of the forces through the articulating component or hydrogel 30, thereby protecting the articulating component or hydrogel 30 from damage. This enables a surgeon to securely compress the implant 10 into the undersized recipient site hole.

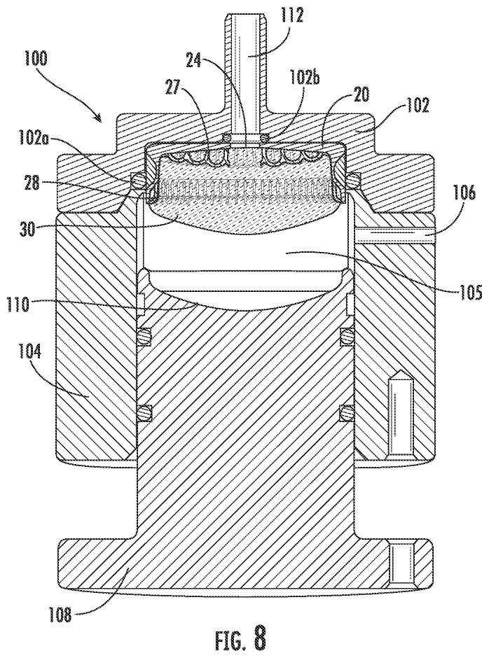

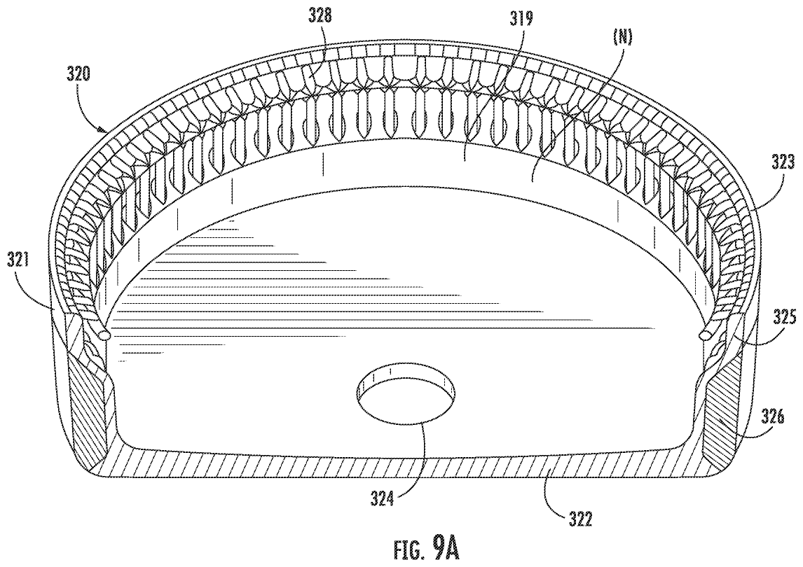

A method of forming the medical implant 10 is shown in FIG. 8. As shown in FIG. 8, a molding assembly 100 is provided for forming the medical implant 10. The molding assembly 100 includes a first portion 102 and a second portion 104. An inlet 106 is defined on a radial or lateral side of the second portion 104 in one aspect. The first portion 102 defines an outlet 112, preferably in a medial region of the first portion 102. The outlet 112 extends in an axial direction from the top of the first portion 102.

A plunger 108 is provided that is configured to be received within an opening of the second portion 104. The plunger 108 includes an engagement surface 110 on an axial end that is configured to form the top surface of the articulating component 30. During formation, the anchor body 20 (including the first and second lattice regions 27, 28, among other features) is placed in the first portion 102. As shown in FIG. 8, the anchor body 20 is inverted within the first portion 102.

A space 105 is defined between first portion 102 and the second portion 104. In one aspect, reinforced bearing supports 102a, 102b are provided and configured to engage the anchor body 20 during the formation process.

A method of forming the medical implant 10 includes initially forming the anchor body 20 separately from the articulating component 30. The anchor body 20 is then placed inside of the first portion 102 such that the bottom wall 22 lays flat on a cup formed by the first portion 102. The aperture 24 in the bottom wall 22 is aligned with the outlet 112. The first portion 102 and the second portion 104 are then joined together. As shown in FIG. 8, the first portion 102 and the second portion 104 have complementary interfaces that join each other. Once the first portion 102 and the second portion 104 are joined together, a flowable material (i.e. hydrogel) is injected via the inlet 106 such that the flowable material fills the space 105. The flowable material completely surrounds the lattice regions 27, 28 of the anchor body 20. Next, the plunger 108 is driven upwards such that the engagement surface 110 presses into the flowable material to form a top bearing surface of the articulating component. As the plunger 108 is driven upwards, air bubbles in the flowable material are driven upwards and out of the assembly via the outlet 112. The articulating component formed from the flowable material subsequently is partially solidified while retaining elasticity to form a cartilage-like surface. In other words, the articulating component is still a flexible and elastic component that can deform but returns to an undeformed state after a load or stress is applied.

Having thus described the presently preferred embodiments in detail, it is to be appreciated and will be apparent to those skilled in the art that many physical changes, only a few of which are exemplified in the detailed description, could be made without altering the inventive concepts and principles embodied therein. It is also to be appreciated that numerous embodiments incorporating only part of the preferred embodiment are possible which do not alter, with respect to those parts, the inventive concepts and principles embodied therein.

The present embodiments and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

What is claimed is:

1. A device configured for use as a medical implant, the device comprising:
an anchor body including:
a perimeter wall defining a rim, and a cavity dimensioned to receive an elastic articulating component;
at least one lattice region arranged at least along an inner surface of the perimeter wall adjacent to the rim; and
an elastic articulating component configured to fill the cavity and attach to the at least one lattice region.

2. The device according to claim 1, wherein the at least one lattice region includes a first lattice region and a second lattice region that are distinct from each other.

3. The device according to claim 2, wherein the first lattice region is arranged along a bottom region of the perimeter wall and the second lattice region is arranged along the inner surface of the perimeter wall adjacent to the rim.

4. The device according to claim 2, wherein the perimeter wall defines an inner surface between the first lattice region and the second lattice region, and wherein the inner surface includes at least one non-lattice region within the cavity.

5. The device according to claim 2, wherein the first lattice region includes a plurality of struts extending upward from a bottom surface of the perimeter wall.

6. The device according to claim 5, wherein a height of the plurality of struts increases in a direction starting from the perimeter wall and radially towards a center of the anchor body.

7. The device according to claim 2, wherein a top surface of the first lattice region defines a radius of curvature between diametrically opposed surfaces of the perimeter wall.

8. The device according to claim 2, wherein the second lattice region is spaced apart from the first lattice region.

9. The device according to claim 2, wherein the first lattice region has a radially innermost edge that is arranged radially outward from an aperture defined by a partially closed bottom surface of the perimeter wall.

10. The device according to claim 2, wherein the first lattice region and the second lattice region are radially offset from each other relative to a central axis of the anchor body.

11. The device according to claim 2, wherein the elastic articulating component is molded with the anchor body, and the elastic articulating component completely surrounds both the first lattice region and the second lattice region.

12. The device according to claim 1, wherein an outer porous layer is arranged on an outer surface of the perimeter wall.

13. The device according to claim 12, wherein a thickness of the outer porous layer is at least three times larger than a second thickness of the perimeter wall in a region of the outer porous layer.

14. The device according to claim 13, wherein the rim has a third thickness which is greater than the second thickness of the perimeter wall in the region of the outer porous layer, and the third thickness is less than the first thickness of the outer porous layer.

15. The device according to claim 2, wherein a top surface of the first lattice region defines a first radius of curvature between diametrically opposed surfaces of the perimeter wall, and wherein a second radius of curvature defined by a top surface of the elastic articulating component is identical to or within 1%-10% of the first radius of curvature.

16. The device according to claim 1, wherein the elastic articulating component is formed from hydrogel.

17. The device according to claim 1, wherein the at least one lattice region is defined along an entirety of the inner surface of the perimeter wall.

18. The device according to claim 1, wherein the at least one lattice region is only defined in a region directly adjacent to the rim.

19. The device according to claim 1, wherein the at least one lattice region is defined as an annular region arranged in a medial area along the interior surface of the perimeter wall, and spaced away from the rim and a bottom surface of the anchor body.

20. A medical implant tool comprising:
an outer sleeve; and
a plunger arranged inside of the outer sleeve such that the plunger is axially moveable relative to the outer sleeve, the plunger defining a collar dimensioned to receive a portion of a medical implant,
wherein the medical implant includes an elastic articulating component molded with an anchor body, the collar is dimensioned to receive a portion of the articulating component, and the collar is configured to engage against a rim defined by the anchor body.

21. A method of inserting a medical implant using a medical implant tool, the method comprising:
providing the medical implant and the medical implant tool,
the medical implant including an elastic articulating component molded with an anchor body, the elastic articulating component formed from hydrogel, and a rim defined at an end of the anchor body;
the medical implant tool including an outer sleeve and a plunger arranged inside of the outer sleeve such that the plunger is axially moveable relative to the outer sleeve, the plunger defining a collar dimensioned to receive a portion of the medical implant;
inserting the medical implant inside an opening in the patient's bone;
engaging the medical implant with the medical implant tool to secure the medical implant within the patient's bone, such that the collar surrounds and receives a portion of the elastic articulating component and the collar engages the rim of the anchor body; and
activating the plunger to engage the rim to press the medical implant within the opening in the patient's bone.
